(19) United States
(12) Patent Application Publication
(10) Pub. No.: US 2007/0197491 A1
(43) Pub. Date: Aug. 23, 2007

(54) Method for treating primary and secondary forms of glaucoma

(75) Inventors: Alan L. Robin, Cockeysville, MD (US); Alan L. Weiner, Arlington, TX (US); David Allen Marsh, Fort Worth, TX (US)

Correspondence Address: ALCON, IP LEGAL, TB4-8, 6201 SOUTH FREEWAY, FORT WORTH, TX, 76134, US

(73) Assignee: ALCON, INC., Hunenberg (CH)

(21) Appl. No.: 11/549,578

(22) Filed: Oct. 13, 2006

Related U.S. Application Data
(60) Provisional application No. 60/726,740, filed on Oct. 14, 2005.
(60) Provisional application No. 60/753,751, filed on Dec. 23, 2005.

(51) Int. Cl. A61K 31/57

(52) U.S. Cl. 514/178

(57) ABSTRACT

Methods and compositions for controlling ocular hypertension associated with (i) primary open angle glaucoma (POAG), (ii) other forms of glaucoma, or (iii) glucocorticoid therapy are disclosed. The methods involve administration of angiostatic agents and other IOP-lowering agents via local injections in the anterior segment of the eye. The most preferred IOP-lowering agents are angiostatic steroids, particularly anecortave acetate, and the most preferred route of administration is an anterior juxtascleral injection or implant. The invention is based in part on the discovery that anterior juxtascleral injections of anecortave acetate are capable of controlling intraocular pressure for sustained periods of from one to several months or more. This result is believed to be attributable to facilitation of access of the anecortave acetate to the trabecular meshwork via the anterior juxtascleral route of administration. This route of administration is also believed to be advantageous for other types of IOP-lowering agents, particularly molecules that cannot readily penetrate the cornea due to size or other physical properties.

### BACKGROUND OF THE INVENTION

This application claims priority from U.S. Ser No. 60/726,740 filed Oct. 14, 2005 and from U.S. Ser. No. 60/753,751 filed Dec. 23, 2005.

### Field of the Invention

This invention relates to methods and compositions for controlling ocular hypertension associated with: (i) primary open angle glaucoma; (ii) other forms of glaucoma; or (iii) glucocorticoid therapy, via local injections of angiostatic agents and other IOP-lowering agents in the anterior segment of the eye, particularly anterior juxtascleral injection.

### DESCRIPTION OF RELATED ART

“Glaucomas” are a group of debilitating eye diseases that are the leading cause of irreversible blindness in the United States in blacks and Hispanics, the second leading cause of blindness in whites in the United States, and a leading cause of blindness in all countries, including both developed and less developed nations. The disease is estimated to affect between 0.4% and 3.3% of all adults over 40 years old (Leske, M. C. et al. (1983); Bengtsson, B. (1989); Strong, N. P. (1992)). Moreover, the prevalence of the disease rises with age to over 6% of those 75 years or older (Strong, N. P., (1992)). It is estimated that by 2010, 60.5 million people worldwide will be affected with open angle glaucoma and angle closure glaucoma, increasing to 79.6 million by 2020. (Quigley and Broman 2006). In all glaucomas, eye pressure lowering is strongly associated with a decrease in the rate of developing the disease and a decrease in the rate of progression towards both disability and blindness. Lowering of the eye's pressure, referred to as “the intraocular pressure” (IOP), is the only known way of successfully treating this disease. We know that for every 1 mm Hg decrease in IOP, the chances of progressive damage decrease by approximately 10%.

The etiology of glaucoma is still the subject of much research in the U.S. and other countries. Although the causes of the disease are still not entirely clear, it is known that the trabecular meshwork of the eye plays a key role in this disease, particularly with respect to the maintenance of fluid dynamics within the eye. Specifically, if the trabecular meshwork does not function as well as it should, this malfunction leads to a relative obstruction of the normal ability of aqueous humor to leave the eye and an elevation of IOP, resulting in progressive visual loss, visual disability and blindness, if not treated appropriately and in a timely fashion.

Elevations of intraocular pressure may also occur as a result of the use of corticosteroids to treat inflammatory diseases. Corticosteroids, particularly glucocorticoids, are currently used to treat a variety of inflammatory diseases. During the last few years, for example, glucocorticoids have been used by the medical community to treat certain disorders of the back of the eye, in particular: Kenalog® (triamcinolone acetonide), Celestone Soluspan® (betamethasone sodium phosphate), Depo-Medrol® (methylprednisolone acetate), Decadron® (dexamethasone sodium phosphate), Decadron L. A.® (dexamethasone acetate), and Aristocort® triamcinolone diacetate). Disorders that have been treated in this way include macular edema following vein occlusion and diabetic retinopathy. Triamcinolone has also been administered following cataract surgery, and administered to eyes with macular edema associated with other vitreo-retinopathies.

These products are commonly administered either topically, via a periocular injection, or an intravitreal injection for the treatment of inflammatory disorders. Because of the lack of efficacious and safe therapies, there is a growing interest in using glucocorticoids for the treatment of, for example, retinal edema and age-related macular degeneration (AMD). Bausch & Lomb and Control Delivery Systems have recently obtained FDA approval for fluocinolone acetonide delivered via an intravitreal implant for the treatment of macular edema. Oculex Pharmaceuticals is studying a dexamethasone implant for persistent macular edema. In addition, ophthalmologists are experimenting with intravitreal injection of triamcinolone acetonide for the treatment of recalcitrant cystic diabetic macular edema and for exudative AMD.

It is known that administration of glucocorticoids to treat inflammatory disorders can also lead to an increase in intraocular pressure. Glucocorticoids can increase the expression of myocilin (MYOC) in the trabecular meshwork, thus increasing myocilin protein secretions. MYOC was originally discovered as a differentially expressed gene and is mapped to glaucoma linkage site GLC1A with mutations found in glaucoma patients. It is expressed in a variety of tissues, including the trabecular meshwork. It is believed that the increase in the expression of MYOC resulting from administration of glucocorticoids causes congestion of the trabecular meshwork, which in turn causes an elevation of IOP. Many authors have documented the frequency and duration of the IOP rise associated with intravitreal triamcinolone injections. The IOP elevation can occur as quickly as 4 days and reach IOPs approaching or exceeding 60 mm Hg (Singh et al. 2004). Usually, the IOP elevation begins 2 to 3 weeks after injection of the steroid (Epstein et al. 1997) and can last 6 to 8 months (Jonas 2003; Jonas 2004). Topical application of IOP-lowering medications has provided some relief from the resulting increase in IOP, but in many cases, does not sufficiently lower the IOP to avoid damage to ocular tissues. Moreover, many patients are prescribed multiple IOP-lowering medications, all of which must be self-administered via topical application, to address their elevated IOP.

Although treatment of such disorders of the back of the eye with glucocorticoids has been effective, one of the most common complications has been a sudden, steroid related elevation of IOP that can occur within days, last at least six months, require medications to lower the elevated IOP, and have serious sight threatening complications due to the continuing presence of the drug in the vitreous or in or around the eye. The etiology of this IOP rise is only partially understood. After the administration of glucocorticoids, there are morphologic and biochemical changes of trabecular meshwork cells. These modifications include increased cell size, and cytoskeletal reorganization, and are believed to be in part due to the significant induction of myocilin mRNA in trabecular meshwork cells.

Patients experiencing elevated IOP as a result of treatment with glucocorticoids are typically prescribed a number of IOP-lowering medications to address this side effect. In many patients, the elevated IOP resulting from glucocorticoid administration tends to persist despite the concurrent use of IOP-lowering medications, which are typically delivered topically. The IOP-lowering medications currently available are frequently unable to adequately control these steroid-induced elevations of IOP. In such cases, surgical intervention with either conventional filtration surgery or shunts may be required. Such surgery carries with it inherent risks that are substantial, especially in the group of subjects who may have multiple additional risks of failure and complications for filtration surgery. Also, many individuals tend to be less than 100% compliant with the prescribed use of their IOP-lowering medications, and this lack of compliance can lead to vision loss.

Treatment regimens currently available for patients exhibiting elevated IOP, regardless of cause, typically include the topical application, from once daily to multiple times per day, of one or multiple eyedrops or pills containing an IOP-lowering compound. Also, pills that decrease the amount of aqueous humor created can be given between two and four times daily. It is estimated that approximately 40% (Ocular Hypertensive Treatment Study; “OHTS”) of those with early glaucoma and approximately 75% (Collaborative Initial Glaucoma Treatment Study; “CIGTS”) of those with more advanced glaucoma require more than one glaucoma medication to adequately lower the IOP.

Both compliance and adjunctive therapy are important problems in glaucoma therapy. Moreover, no current intraocular pressure (IOP) lowering medication can be routinely given at intervals greater than 24 hours per dose. All current glaucoma therapies are given either topically or orally and do not routinely yield an additional 25% decrease in IOP lowering when added to another IOP-lowering medication. In addition, in a significant number of patients, it is not possible to control IOP adequately via the topical application of one or more existing IOP-lowering medications. In order to achieve adequate control of IOP in such patients, conventional glaucoma filtration surgery or shunts are frequently necessary. There is a significant need for an improved means for controlling IOP in these patients without resorting to surgery. The present invention address this need by providing a means for achieving adequate control of IOP in such patients, via the use of a new route of administration, particularly anterior juxtascleral injections of anecortave acetate and other angiostatic agents.

Many individuals are unable to take eye drops (Sleath et al., 2000) and pills have so many associated adverse events associated with them that over 50% of patients are unable to tolerate them, even for short term usage. Additionally, many patients don't comply with the prescribed treatment regimen for topical medication usage. It has been shown that, the more complex the medical regimen, the less likely a patient is to adhere to the therapy (Robin and Covert, 2005). The effectiveness of the prescribed treatment regimen and the benefit to the patient is diminished as the patient does not appropriately take his or her medication. Moreover, many patients, once diagnosed and prescribed medications, fail to return for routine follow up (Nordstrom et al., 2005).

In a review of literature studying patient compliance to treatment regimens, it was found that eye disorders (i.e., glaucoma), were included in the five conditions falling at the bottom of the medical condition compliance list. (DiMatteo 2004) It is believed that the low compliance rate for patients with eye disorders may, in part, be related to variations in treatment regimen, including the number of prescribed daily doses, the number of medications prescribed, the route of administration, methods of compliance assessment and duration of the compliance study period. Some literature has estimated compliance to eye drop regimens to range from 40% to 78% (Gurwitz et al. 1998; Spooner et al. 2002; Lee et al. 2000; Patel and Spaeth 1995; Claxton et al. 2001). Whatever the cause, non-compliance leads to inadequate control of intraocular pressure and increased loss of visual field.

There is a need for a management regimen for treating elevated intraocular pressure, whether resulting from a form of glaucoma or administration of corticosteroids, that provides long-lasting efficacy and lowering of IOP to the patient without requiring daily self-administration of medication. A therapy that can accommodate individuals who may not refill medications and miss multiple follow ups is needed. The methods and compositions of this invention meet that need.

### REFERENCES

The following references may be referred to for further background information. To the extent that these references provide exemplary procedural or other details supplementary to those set forth herein, such contents of the references are specifically incorporated herein by reference.

United States Patents
U.S. Pat. No. 5,407,926
U.S. Pat. No. 5,679,666
U.S. Pat. No. 5,770,589
U.S. Pat. No. 5,770,592
U.S. Pat. No. 5,972,922

Foreign Publications
WO 00/02564

Books
Grierson I and Calthorpe C M, “Characteristics of Meshwork Cells and Age Changes in the Outflow System of the Eye: Their Relevance to Primary Open Angle Glaucoma.” In Mills K B (ed). Glaucoma. Proceedings of the Fourth International Symposium of the Northern Eye Institute, Manchester, UK, New York, Pergamon: pp. 12-31 (1988).
Hernandez M and Gong H, “Extracellular matrix of the trabecular meshwork and optic nerve head.” In Ritch R., Shields, M. B., Krupin, T. (eds). The Glaucomas, 2^{nd} ed. St Louis: Mosby-Year; pp. 213-249 (1996).
Lutjen-Drecoll E., Rohen J. W., “Morphology of aqueous outflow pathways in normal and glaucomatous eyes,” in Ritch R., Shields, M. B., Krupin, T. (eds). The Glaucomas, 2^{ed}. St Louis: Mosby-Year; pp. 89-123 (1996).

Other Publications
Bengtsson, B., Br. J. Ophthalmol. 73:483-487 (1989).
Clark et al., “Ocular angiostatic agents,” Exp. Opin. Ther. Patients 10(4):427-448 (2000)
Epstein et al. (1997).
Hernandez M R, Andrzejewska W M, Neufeld A H, “Changes in the extracellular matrix of the human optic nerve head in primary open-angle glaucoma,” Am. J. Ophthalmol. 109:180-188 (1990).
Hernandez M R, Pena J D, “The optic nerve head in glaucomatous optic neuropathy,” Arch Ophthalmol. 115:389-395 (1997).
Jonas et al., “Intraocular pressure after intravitreal injection of triamcinolone acetonide”, Br. J. Ophthalmol, 87:24-27 (2003).
Jonas et al., “Safety of Intravitreal High-dose Reinjections of Triamcinolone Acetonide”, American Journal of Ophthalmology, 138(6):1054-1055 (2004).
Kerrigan L A, Zack D J, Quigley H A, Smith S D, Pease M E, “TUNEL-positive ganglion cells in human primary open-angle glaucoma,” Arch. Ophthalmol. 115:1031-1035 (1997).
Leske M C, et al., “The Epidemiology Of Open-Angle Glaucoma: A Review”, American Journal of Epidemiology, 118(2):166-191 (1983).
Nordstrom, Friedman, et al., AJO, 140:598-606 (2005).
Morrison J C, Dorman-Pease M E, Dunkelberger G R, Quigley H A, “Optic nerve head extracellular matrix in primary optic atrophy and experimental glaucoma,” Arch. Ophthalmol. 108:1020-1024 (1990).
Quigley, H. A., and Broman, A. T., “The number of people with glaucoma worldwide in 2010 and 2020,” Br J. Ophthalmol. 90:262-267 (2006).
Quigley H A, Nickells R W, Kerrigan L A, Pease M E, Thibault D J, Zack D J, “Retinal ganglion cell death in experimental glaucoma and after axotomy occurs by apoptosis,” Invest. Ophthalmol. Vis. Sci. 36:774-786 (1995).
Quigley H A, “Neuronal death in glaucoma,” Prog. Retin. Eye Res. 18:39-57 (1999).
Quigley H A, McKinnon S J, Zack D J, Pease M E< Kerrigan-Baumrind L A, Kerrigan D F, Mitchell R S, “Retrograde axonal transport of BDNF in retinal ganglion cells is blocked by acute IOP elevation in rats,” Invest. Ophthalmol. Vis. Sci. 41:3460-3466 (2000).
Rohen J W, “Why is intraocular pressure elevated in chronic simple glaucoma? Anatomical considerations.” Ophthalmology 90:758-765 (1983).
Robin and Covert, “Does Adjunctive Glaucoma Therapy Affect Adherence to the Initial Primary Therapy?”, American Academy of Ophthalmology, 112(5):863-868 (2005).
Singh et al., “Early Rapid Rise in Intraocular Pressure After Intravitreal Triamcinolone Acetonide Injection”, American Journal of Ophthalmology, 138(2):286-287 (2004).
Sleath et al., “Patient Expression of Complaints and Adherence Problems with Medications During Chronic Disease Medical Visits”, Journal of Social and Administrative Pharmacy, 17(2):71-80 (2000).
Strong, N. P., Ophthal. Physiol. Opt. 12:3-7 (1992).
Varma R. et al., “Prevalence of Open-Angle Glaucoma and Ocular Hypertension in Latinos”, American Academy of Ophthalmology, 111(8):1439-1448 (2004).

### SUMMARY OF THE INVENTION

The invention encompasses methods and compositions for treating glaucoma, or for controlling elevated intraocular pressure (IOP), by administering a medication for treating glaucoma to the anterior segment of a patient's eye, preferably via anterior juxtascleral administration of drug depots. In certain embodiments, the medication administered will be an IOP-lowering medication. Preferably, the medication to be administered according to the methods of the present invention will be an angiostatic agent, such as an angiostatic cortisene.

In one preferred embodiment, the invention provides a method for lowering intraocular pressure in a patient having a form of glaucoma. According to the methods of the invention, a composition comprising an IOP-lowering agent is administered to a patient suffering from elevated intraocular pressure via anterior juxtascleral depot administration. The IOP-lowering agent may be any agent known to cause a decrease in intraocular pressure, such as a carbonic anhydrase inhibitor, a beta blocker, an alpha agonist, a serotonergic, ethacrynic acid, a miotic, a prostaglandin analog, or an angiostatic agent. Preferably, the agent will be an angiostatic agent, such as an angiostatic cortisene.

In another preferred embodiment, the invention provides a method for lowering intraocular pressure in a patient having elevated intraocular pressure, or at risk for developing elevated intraocular pressure, resulting from intravitreal injection or other administration of a glucocorticoid. The method of the invention includes administering to a patient, who has had or who will have an administration of a glucocorticoid for the treatment of vitreoretinal disorders or other disorders of the back of the eye, a composition comprising a therapeutically effective amount of an IOP-lowering agent. Typically, administration of the IOP-lowering agent will occur prior to, subsequent to, or simultaneously with intravitreal injection of the glucocorticoid.

While it is envisioned that the glucocorticoid may be any glucocorticoid used to treat retinal disorders or other disorders of the back of the eye or to treat inflammation resulting from surgical procedures, in certain preferred embodiments, the glucocorticoid will be triamcinolone acetonide. In other preferred embodiments, the glucocorticoid will be fluocinolone acetonide, dexamethasone, prednisolone or lotoprednisol, or others.

In general, the methods of the invention include administering to a patient in need thereof, a composition comprising a therapeutically effective amount of an IOP-lowering medication. The agent is preferably administered by anterior juxtascleral depot administration. Other methods of administering the IOP-lowering agent include anterior subtenon administration, anterior subconjunctival injection, anterior juxtascleral depot administration, and anterior implant.

While it is contemplated that any agent that is capable of controlling or preventing IOP elevations will be useful in the methods of the invention, the preferred agent is an angiostatic agent. The preferred angiostatic agent for use in the methods of the present invention is 4,9(11)-pregnadien-17∝,21-diol-3,20-dione-21-acetate, also known as anecortave acetate, or its corresponding alcohol, 4,9(11)-pregnadien-17α,21-diol-3.20-dione, also known as anecortave desacetate.

### BRIEF DESCRIPTION OF THE DRAWINGS

The drawings that accompany the present application, which are briefly described below, form part of the present specification and are included to further demonstrate certain aspects of the present invention. The invention may be better understood by reference to these drawings in combination with the detailed description of specific embodiments presented herein.

FIG. 1 illustrates the anterior juxtascleral depot delivery method of the present invention. A suspension containing an IOP-lowering medication is administered via anterior juxtascleral depot administration in the inferior or inferior temporal quandrant of the patient's eye. FIG. 1A illustrates the procedure at the beginning of administration of the composition. FIG. 1B illustrates the procedure after administration of the desired amount of the composition.

FIG. 2 illustrates the decrease in IOP over eight months of six patients injected with anecortave acetate in the anterior segment of the eye, as described in Example 2.

FIG. 3 illustrates the decrease in IOP over time of six patients injected with anecortave acetate in the anterior segment of the eye subsequent to administration with glucocorticoid, as described in Example 3.

FIG. 4 illustrates the decrease in IOP over time of Dutch Belted rabbits having elevated IOP injected in the anterior segment with a carbonic anhydrase inhibitor, as described in Example 4.

FIG. 5 illustrates the decrease in IOP over time of Dutch Belted rabbits having elevated IOP injected in the anterior segment with a prostaglandin analog, as described in Example 5.

### DETAILED DESCRIPTION OF PREFERRED EMBODIMENTS

The present invention is based, in part, on a discovery that local injections of IOP-lowering agents in the anterior segment of the eye, e.g., via anterior juxtascleral depot administration, of an IOP-lowering agent, is more effective at addressing elevated IOP associated with glaucoma or resulting from administration of glucocorticoids than currently known methods of treatment. The advantages of the delivery methods of the present invention, where the medication migrates to the area anterior to the trabecular meshwork, include: 1) allowing for the use of medications that might not be effective if delivered topically, as eye drops; and 2) providing sustained and long-term duration of action, obviating compliance issues.

The present is further based in part on a discovery that, due to the long-lasting nature of depot delivery of anecortave acetate, intraocular administration of this, or other relatively insoluble IOP-lowering drugs via local injections in the anterior segment, particularly anterior juxtascleral injections, are capable of providing sustained control of IOP elevations associated with glaucoma or from administration of glucocorticoids.

The IOP-lowering agent may be any agent administered for the purpose of decreasing IOP in a patient suffering from elevated IOP. Alternatively, the IOP-lowering agent may be a large molecule that has IOP-lowering activity, but that would not be therapeutically effective following topical application to the eye, due to limited corneal penetration. In preferred aspects, the IOP-lowering agent will be a relatively insoluble agent, capable of being formulated for anterior juxtascleral depot administration, so as to provide for control of IOP over sustained periods of one month or more, preferably three months or more, and most preferably six months or more. IOP-lowering agents useful in the methods of the invention include angiostatic agents, carbonic anhydrase inhibitors, alpha 1 antagonists, alpha 2 agonists, beta blockers, serotonergics, ethacrynic acid, miotics, or prostaglandin analogs. The preferred agent for use in the methods of the invention is an angiostatic agent, such as an angiostatic cortisene.

Agents which inhibit angiogenesis are known by a variety of terms such as angiostatic, angiolytic or angiotropic agents. For purposes of this specification, the term “angiostatic agent” means compounds which can be used to inhibit angiogenesis, but that lack the glucocorticoid activity associated with steroids. The most preferred compound for use in the methods of the invention is the angiostatic cortisene, 4,9(11)-pregnadien-17∝,21-diol-3,20-dione-21-acetate, also known as anecortave acetate.

Anecortave acetate is a cortisene and an analog of cortisol acetate. Among the modifications to the steroid backbone are the removal of the 11-hydroxyl group, introduction of the C9-11 double bond and an addition of a 21-acetate group. As a result of these modifications anecortave acetate lacks the typical anti-inflammatory and immunosuppressive properties of glucocorticoids. Anecortave acetate downregulates trabecular meshwork myocilin expression. Using cultured trabecular meshwork cells, Clark et al. (2000) demonstrated the inhibition by anecortave acetate of dexamethasone induced myocilin expression. Clark discusses the finding that topical administration of anecortave acetate decreases the IOP elevation associated with the topical administration of dexamethasone in rabbits. However, as indicated above, many patients don't comply with the prescribed treatment regimen for topical medication usage.

Examples of possible specific IOP-lowering agents include beta-blockers (e.g., timolol, betaxolol, levobetaxolol, carteolol, levobunolol, and propranolol), carbonic anhydrase inhibitors (e.g., brinzolamide and dorzolamide), alpha-1 antagonists (e.g., nipradolol), alpha-2 agonists (e.g. iopidine and brimonidine), miotics (e.g., pilocarpine and epinephrine), prostaglandin analogs (e.g., latanoprost, travoprost and unoprostone), hypotensive lipids (e.g., bimatoprost and compounds set forth in U.S. Pat. No. 5,352,708), neuroprotectants (e.g., memantine), serotonergics [e.g., 5-HT₂ agonists, such as S-(+)-1-(2-aminopropyl)-indazole-6-ol)], anti-angiogenesis agents (e.g., anecortave acetate), and ethacrynic acid. The ophthalmic drug may be present in the form of a pharmaceutically acceptable salt, such as timolol maleate, brimonidine tartrate or sodium diclofenac. The compositions of the present invention may also include combinations of ophthalmic drugs, such as combinations of (i) a beta-blocker selected from the group consisting of betaxolol and timolol, (ii) a prostaglandin analog selected from the group consisting of latanoprost, 1,5-keto latanoprost, travoprost, bimatoprost, and unoprostone isopropyl, and (iii) an angiostatic steroid (e.g., anecortave acetate) in combination with a prostaglandin analog and/or any of the other IOP-lowering agents identified above.

According to the methods of the present invention, a relatively insoluble IOP-lowering composition, is administered by anterior juxtascleral depot administration, in order to control elevated IOP associated with glaucoma or resulting from treatment with glucocorticoids. In certain embodiments of the present invention, a glucocorticoid is administered intraocularly to treat disorders of the back of the eye, such as ocular angiogenesis, edema, or diabetic retinopathy, or to treat inflammation resulting from surgical procedures, such as vein occlusion or cataract surgery. An IOP-lowering agent, such as anecortave acetate, is administered to the eye of the patient via anterior juxtascleral depot administration. The IOP-lowering agent may be administered prior to, concurrently with, or subsequent to, administration of the glucocorticoid. It is envisioned that the administrations of triamcinolone and the IOP-lowering agent could take place minutes, hours, days, weeks, or even months apart.

Although the IOP-lowering agent used in the methods of the present invention will typically be administered via anterior juxtascleral depot administration, the agent may alternatively be administered via anterior subtenon's administration, anterior subconjunctival injection, anterior implant and combinations thereof.

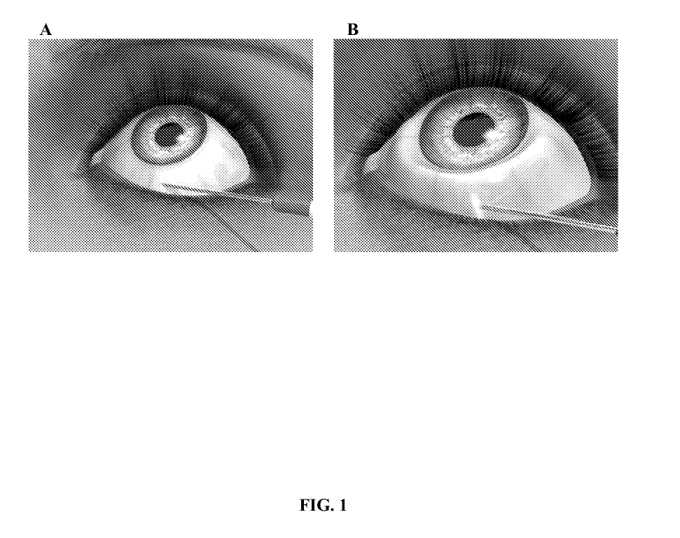

The anterior juxtascleral depot route of administration is typically performed as follows: A composition containing the IOP-lowering agent to be administered is transferred to a syringe using sterile technique. A 30 gauge needle is attached to the syringe. The desired amount of the composition is placed as an anterior juxtascleral depot in the inferior or inferior temporal quadrant of the eye. See FIG. 1 for placement of the anterior juxtascleral depot.

Administering an IOP-lowering agent via anterior juxtascleral depot administration, according to the methods of the present invention, will typically provide a reduction of IOP for a period of from about 2 months to 12 months, preferably from about 3 months to 8 months, more preferably for at least 6 months. The amount of the IOP-lowering agent in the composition delivered via anterior juxtascleral depot administration will typically be from about 0.5 mL to about 1 mL, with the maximum amount of drug to be delivered being from about 250 mg (for delivery of 0.5 mL) to about 500 mg (for delivery of 1 mL). Alternatively, the percent of the IOP-lowering agent in the composition will generally be up to about 50 weight percent. Determination of maximum injectable percent suspension will depend on particle size of the IOP-lowering agent and other factors well known to the skilled artisan.

Likewise, formulating the composition to achieve the optimal rate needed to achieve therapeutic tissue levels will be defined by pharmacokinetics and pharmacology and other factors well known to the skilled artisan. In general, solubility and/or drug diffusion from the particle should be no less than the rate needed to achieve therapeutic tissue level. As will be readily apparent to the skilled artisan, any level of water solubility for the drug in suspension is possible if the following factors are considered: 1) the minimum amount solubilized and released per day should correspond to what is needed for efficacy; 2) the amount injected should be sufficient to have the duration of action desired; 3) the limit for injectability should not be exceeded; and 4) rates above the minimum rate needed to meet the desired duration of action do not adversely affect safety.

In preferred aspects of the present invention, anecortave acetate is administered via anterior juxtascleral depot administration, in order to allow it to more efficiently function to lower the elevated IOP associated with OAG or resulting from administration of glucocorticoids. The amount of the anecortave acetate administered by anterior juxtascleral depot administration will generally be from about 1 mg to about 60 mg. Preferably, the amount of anecortave acetate administered to the patient will be from about 3 mg to about 30 mg; from about 12 mg to about 27 mg; or from about 21 mg to about 27 mg. The most preferred dosage for administration is 24 mg of anecortave acetate. Alternatively, the preferred concentration of the angiostatic agent in the composition administered via the methods of the invention is from 0.005 to 5 weight percent.

The compositions for use in the methods of the invention are formulated in accordance with methods known in the art, depending on the particular route of administration required. The composition will typically be a suspension containing a therapeutic amount of a relatively insoluble IOP-lowering agent, such as a large molecule that would not otherwise penetrate the cornea if delivered topically, or any known IOP-lowering agent. Such composition will generally be formulated for anterior subtenon administration, anterior subconjunctival injection, anterior juxtascleral depot administration, anterior implant, and combinations thereof. In other embodiments, the composition may be a gel or tablet formulated for administration as a depot or implant.

The concentration of IOP-lowering agents to be used in the methods of the invention will be routinely determined by the skilled artisan based upon the type of compound, the patient, the type of composition, and other factors. Preferably, the composition will have a formulation set forth in U.S. Pat. Nos. 5,972,922; 5,679,666; or 5,770,592, each incorporated herein by reference. Most preferably, the composition will have the formulation set forth in Example 1.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

The following formulation is representative of formulations suitable for use in the methods of the present invention.

| Ingredient | Amount (wt %) |
|---|---|
| 4,9(11)-Pregnadien-17α,21-diol-3,20- | 0.1-5.0 |
| dione-21-acetate | |
| Tyloxapol | 0.01-0.05 |
| HPMC | 0.5 |
| Benzalkonium chloride | 0.01 |
| Sodium chloride | 0.8 |
| Edetate Disodium | 0.01 |
| NaOH/HCl | q.s. pH 7.4 |
| Purified Water | q.s. 100 mL |

### EXAMPLE 2

A single administration of approximately 24 mg of anecortave acetate was given via subtenon's administration in the inferior or inferior temporal quadrant to 5 eyes of 6 patients with primary open angle glaucoma.

Methods:

An investigator IND and IRB approval was obtained. All patients gave informed consent. An inferior AJD was given under topical anesthesia and we followed patients at weeks 1, 2, & 4; and monthly thereafter. Prior glaucoma medications were not changed throughout study.

Results:

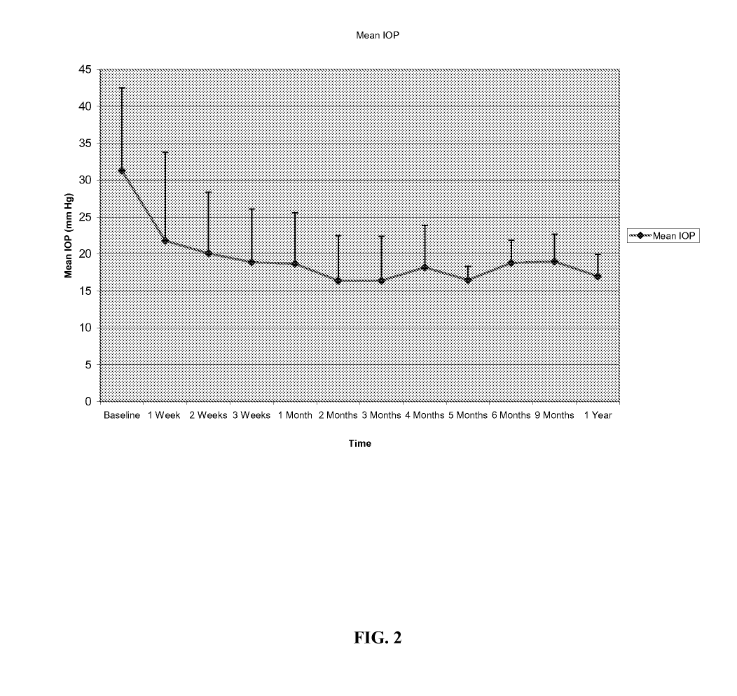

Six subjects with glaucoma and IOP≧23 mmHg (POAG [4], PDS [1], PXF [1]) mean age 59+/−8 years. Mean C/D ratio 0.8+/−0.2. Prior glaucoma medications included prostaglandins, beta blockers and/or alpha agonists (four on 1, one on 3, and one on 4). Mean pretreatment IOP was 31.3+/−11.3 mmHg. Five of six patients had a >25% IOP decrease at 3 months with a mean IOP of 16.4+/−6 mm Hg and a mean 10.8+/−7.0 mmHg (38.5%+/−21%) IOP decrease. (See FIG. 2) No clinically significant adverse events occurred. Patients were followed for twelve (12) months. The IOP-lowering effects peaked at approximately one month. The duration of effectiveness of the anecortave acetate was at least twelve (12) months.

Discussion:

The above-discussed results demonstrate a long term effect from an anterior juxtascleral deposition of anecortave acetate. Treatment with prior glaucoma medications was discontinued for two patients due to the surprising IOP-lowering effects of the juxtasclerally administered anecortave acetate. This new method of treatment obviates problems with eye drops and many issues with compliance. Clinically meaningful additional medium-term IOP reduction is possible with a single anterior juxtascleral depot injection of anecortave acetate, much more than presently obtained with any currently available adjunctive medications.

### EXAMPLE 3

A single administration of approximately 24 mg of anecortave acetate was given via subtenon's administration in the inferior or inferior temporal quadrant to 8 eyes of 7 patients with glaucoma caused by one or more intravitreal injections of glucocorticoids (the number of injections per eye ranged from 1-8). All patients were on maximal tolerated medical therapy for glaucoma and continued on their pre-study medications for the duration of the study. As shown in Table 2 below, the average pre-treatment IOP was 40.125+/−10.8 mmHg. This administration of anecortave acetate resulted in IOP reductions ranging from 29% to 51%, with IOP reductions lasting at least 6 months without adverse events, thereby avoiding glaucoma filtration surgery in 75% of the patients.

TABLE 2

| | Baseline | 1 week | 2 weeks | 3 weeks | 4 weeks | 2 mon | 3 mon | 4 mon | 5 mon | 6 mon | 6.2 mon |
|---|---|---|---|---|---|---|---|---|---|---|---|
| Patient 1* | 57 | 34 | 32 | 30 | 31 | 30 | | | | | |
| Patient 2 | 35 | 24 | 19 | 17 | 14 | 16 | 18 | 16 | 17 | 15 | |
| Patient 3 (OS) | 34 | 24 | 23 | 19 | 21 | 19 | 21 | 23 | 21 | 19 | |
| Patient 3 (OD) | 40 | 23 | 19 | 19 | 19 | 23 | 19 | 21 | 22 | 19 | |
| Patient 4 R | 38 | 34 | 28 | 37 | 30 | | | | | | |
| Patient 5 | 34 | 30 | 35 | | 36 | | | | | | |
| Patient 6 | 41 | 30 | | | 30 | 30 | 32 | 17 | | | |
| Patient 7 | 42 | | 28 | | 28 | 24 | 18 | | 15 | 19 | 26 |
| Mean | 40.125 | 28.42857 | 24.8 | 24.4 | 26.1 | 22 | 19.3 | 20 | 20 | 17.6 | |
| | 6.5 mon | 7 mon | 8 mon | 9 mon | 12 mon | 13 mon | 14 mon | 15 mon | 17 mon | 18 mon | 20 mon |
| Patient 1* | | | | | | | | | | | |
| Patient 2 | | 17 | | 15 | 15 | 18 | | | | | 16 |
| Patient 3 (OS) | | 18 | 31 | 19 | 22 | 22 | 25 | 26 | 19 | 19 | |
| Patient 3 (OD) | | 18 | 22 | 18 | 22 | 19 | 26 | 22 | 18 | 17 | |
| Patient 4 R | | | | | | | | | | | |
| Patient 5 | | | | | | | | | | | |
| Patient 6 | | | | | | | | | | | |
| Patient 7 | 38 | | | | | | | | | | |
| Mean | | 17.6 | 26.5 | 17.3 | 19.6 | 19.6 | 25.5 | 24 | 18.5 | 18 | |

*Patient one was discontinued from the study after two months despite a 27 mm Hg decrease in IOP as the patient had almost total disc cupping and it was felt that it would be best to further lower her IOP surgically.

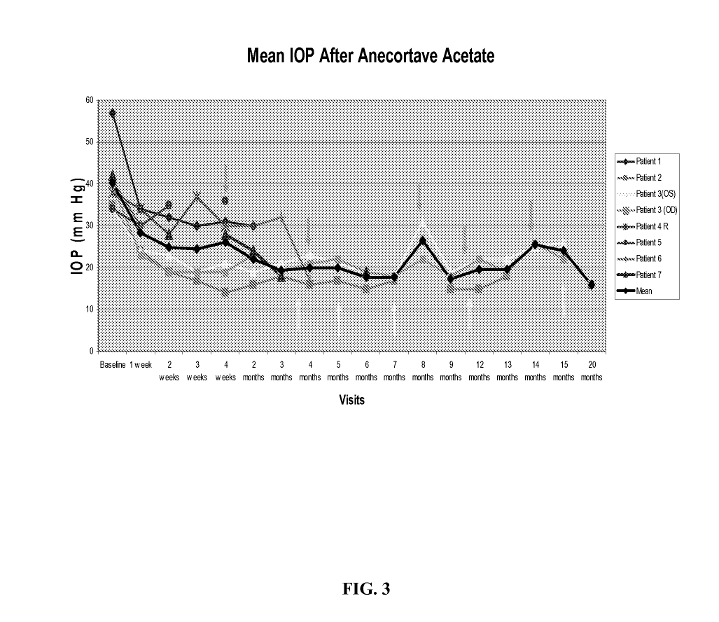

In this group of eight eyes, three of which had prior intraocular surgery, and all of which were on at least three different types of glaucoma medications (maximum 6 medications and a mean of 4.1 different drug classes) the IOP decrease was seen as early as one week (mean 11.4 mm Hg), and appeared to reach a maximum decrease at three weeks (mean 16.4 mm Hg). FIG. 3 illustrates the decrease in IOP observed in these patients for twenty months. All eyes had marked IOP decrease.

In one eye, despite a 45% decrease, the resultant IOP of 30 mm Hg at 2 months was inadequate for the patient's optic nerve, and filtration surgery was necessary. In two additional eyes, the IOP decrease was insufficient to prevent surgical intervention. In the remaining five eyes, anecortave acetate reversed the IOP elevation due to triamcinolone, and prevented a recurrence of elevated IOP for up to 12 months, thus obviating the need for further surgery.

Despite being on multiple glaucoma medications, the mean IOP decrease ranged from 29% to 51% during this 12 month period. The IOP decreases that were observed were much higher than one normally sees by adding another glaucoma medication. Additionally, the IOP lowering effect persisted for several months.

### EXAMPLE 4

Eyes of Dutch Belted rabbits having elevated IOP were injected in the anterior segment with a carbonic anhydrase inhibitor.

Methods:

Baseline IOP was measured daily for 5 days and averaged. Seven rabbits received one anterior sub-Tenon's capsule administration of 800 μl of a non-optimized 1% ophthalmic suspension of brinzolamide (AZOPT®). Seven rabbits received a 1% ophthalmic suspension of brinzolamide (AZOPT®) delivered topically once per day for seven days. Seven rabbits received one anterior sub-Tenon's administration of 800 μl of BSS®. IOP was monitored daily at 2 hours after topical drops were administered, for seven days, and weekly thereafter until IOP measurements remained the same as baseline for two measurements.

Results:

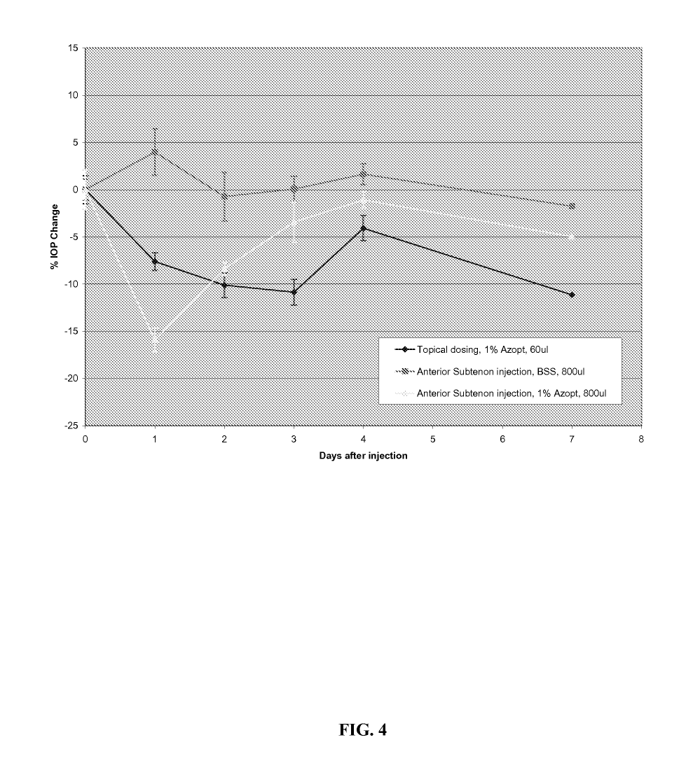

IOP was not significantly changed from baseline in rabbits receiving one injection of a BSS® vehicle solution at the beginning of the study. Mean pretreatment IOP was 27.41 mm Hg. Mean change in IOP for this group was +0.18 mm Hg. Rabbits receiving either daily topical administration or subtenon's injection of brinzolamide experienced sustained lowering of IOP. For the topical administration group mean pretreatment IOP was 28.37 mm Hg. Mean change in IOP for this group was −2.48 mm Hg. In this group a maximum of 11.1% IOP lowering from baseline was observed during the evaluation period. For the group receiving one anterior subtenon's administration of a 1% brinzolamide ophthalmic suspension the mean pretreatment IOP was 27.44 mm Hg. Mean change in IOP for this group was −1.85 mm Hg. The maximum percent IOP lowering observed during the evaluation period for the sub-Tenon's injection group was 15.9%. (See FIG. 4).

Discussion:

The mean percent IOP change from baseline was statistically lower at all points in both the topical administration group and the subtenon's injection group, compared to the BSS® control over 7 days with peak levels observed within the first 3 days. Longer duration of action of the brinzolamide suspension from the subtenon's capsule could be achieved using higher concentration suspensions (e.g. 5% or 10%) or via encapsulation in sustained release dose forms such as microspheres.

### EXAMPLE 5

Eyes of Dutch Belted rabbits having elevated IOP were injected in the anterior segment with a prostaglandin analog.

Methods:

Seven rabbits received anterior subtenon's administration of 1 ML of a microsphere suspension containing 1% prostaglandin analog. Seven rabbits received anterior subtenon's administration of 1 mL of a microsphere suspension containing 2.5% prostaglandin analog. Seven rabbits received anterior subtenon's administration of empty placebo microspheres. IOP was monitored daily for the first week, then once per week thereafter until IOP was back to baseline.

Results:

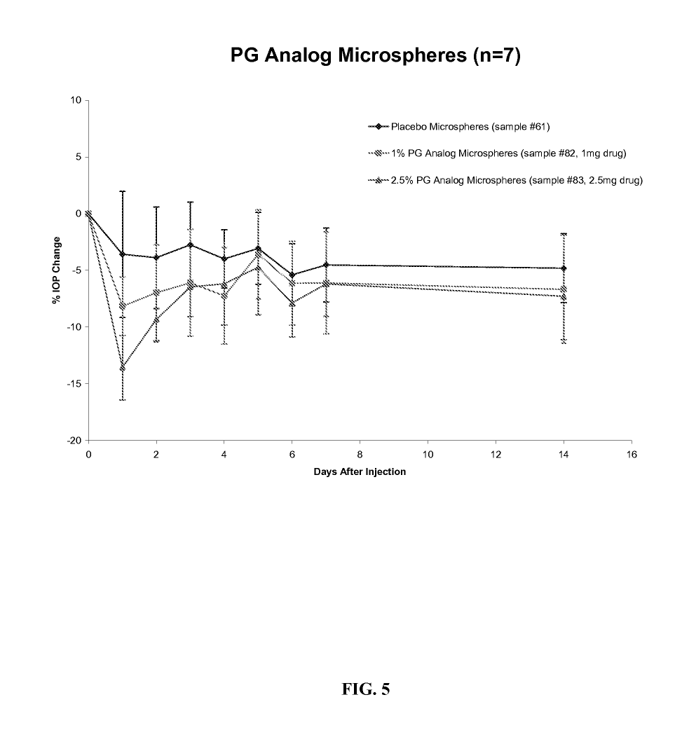

Animals receiving either 1% or 2.5% prostaglandin analog microsphere suspensions exhibited sustained percent decrease in IOP from baseline for a minimum of 4 days. In both prostaglandin analog microsphere suspension groups, percent IOP change was lower than placebo microspheres at all points over 14 days. The IOP lowering appeared to be dose dependant with the group receiving the 2.5% prostaglandin analog suspension showing greater effect (mean IOP decrease=−1.97 mm Hg) than the group receiving the 1% prostaglandin analog suspension (mean IOP decrease=−1.63 mm Hg). The percent IOP decrease in the 1% suspension group ranged from 3.58% to a maximum lowering of 8.17% over the 14 days. The percent IOP decrease in the 2.5% suspension group ranged from 4.73% to a maximum lowering of 13.54% over the course of the 14 days. The group receiving placebo suspension exhibited mean IOP decrease of only 1.0 mm Hg and the maximum percent IOP lowering observed with the placebo during the 14 days was only 5.39% (See FIG. 5)

Discussion:

Animals receiving subtenon's injection of empty microspheres showed a maximum placebo effect of 5.39% IOP change from baseline. However, when injected with microsphere suspensions loaded with either 1% or 2% concentrations of prostaglandin analogs, greater maximum percent IOP lowering was observed (8.17% and 13.54%, respectively). This dose dependant effect was sustained for a minimum of 4 days with some greater residual IOP lowering effect (percent of baseline) observed for the drug loaded microspheres compared to empty microspheres over 14 days.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and structurally related may be substituted for the agents described herein to achieve similar results. All such substitutions and modifications apparent to those skilled in the art are deemed to be within the spirit, scope and concept of the invention as defined by the appended claims.

We claim:

1. A method for controlling intraocular pressure in a human patient, said method comprising administering to said patient a composition comprising a therapeutically effective amount of an IOP-lowering agent, wherein said administration is by a method selected from the group consisting of anterior subtenon administration, anterior subconjunctival injection, anterior juxtascleral depot administration, anterior implant, and combinations thereof.

2. The method of claim 1, wherein the administration is by anterior juxtascleral depot administration.

3. The method of claim 2, wherein the IOP-lowering agent is selected from the group consisting of angiostatic agents, carbonic anhydrase inhibitors, miotics, beta blockers, alpha 1 antagonists, alpha 2 agonists, serotonergics, ethacrynic acid and prostaglandin analogs.

4. The method of claim 1, wherein the IOP-lowering agent comprises an angiostatic agent.

5. The method of claim 4, wherein the angiostatic agent is selected from the group consisting of 4,9(11)-pregnadien-17∝,21-diol-3,20-dione-21-acetate and 4,9(11)-pregnadien-17∝,21-diol-3,20-dione.

6. The method of claim 5 wherein the angiostatic agent is present in the composition at a concentration of 0.005 to 5.0 weight percent.

7. The method of claim 2, wherein the IOP-lowering agent comprises an angiostatic agent and the amount of the angiostatic agent administered is from about 3 mg to about 30 mg.

8. The method of claim 7, wherein the angiostatic agent is anecortave acetate.

9. The method of claim 8, wherein the amount of the angiostatic agent administered is about 24 mg.

10. The method of claim 1, wherein said patient has elevated intraocular pressure, or is at risk for developing elevated intraocular pressure, resulting from administration of a glucocorticoid, and wherein administration of the IOP-lowering agent occurs prior to or subsequent to administration of the glucocorticoid.

11. The method of claim 10, wherein the IOP-lowering agent is administered subsequent to administration of the glucocorticoid.

12. The method of claim 11, wherein the IOP-lowering agent is administered within an hour after administration of the glucocorticoid.

13. The method of claim 11, wherein the angiostatic agent is administered one to five days after administration of the glucocorticoid.

14. The method of claim 11, wherein the angiostatic agent is administered within one week after administration of the glucocorticoid.

15. The method of claim 11, wherein the angiostatic agent is administered one week to eight weeks after administration of the glucocorticoid.

16. The method of claim 11, wherein the angiostatic agent is administered within three months after administration of the glucocorticoid.

17. The method of claim 1, wherein said patient has primary open angle glaucoma.

18. A composition comprising a therapeutically effective amount of an IOP-lowering agent for controlling intraocular pressure in a patient, where said composition is administered by a method selected from the group consisting of anterior subtenon administration, anterior subconjunctival injection, anterior juxtascleral depot administration, anterior implant, and combinations thereof.
